(19) United States
(12) Patent Application Publication
(10) Pub. No.: US 2025/0069225 A1
(43) Pub. Date: Feb. 27, 2025

(54) SYSTEMS AND METHODS FOR MYOCARDIAL INFORMATION DETERMINATION

(71) Applicant: SHANGHAI UNITED IMAGING HEALTHCARE CO., LTD., Shanghai (CN)

(72) Inventors: Yihuan LU, Shanghai (CN); Yizhang ZHAO, Shanghai (CN); Qing YE, Shanghai (CN); Yun DONG, Shanghai (CN)

(73) Assignee: SHANGHAI UNITED IMAGING HEALTHCARE CO., LTD., Shanghai (CN)

(21) Appl. No.: 18/945,480

(22) Filed: Nov. 12, 2024

Related U.S. Application Data
(63) Continuation of application No. PCT/CN2023/139899, filed on Dec. 19, 2023.

(30) Foreign Application Priority Data
Dec. 20, 2022 (CN) 202211640229.2

(51) Int. Cl. G06T 7/00; G06T 7/11

(52) U.S. Cl. CPC G06T 7/0012; G06T 7/11; G06T 2207/10104; G06T 2207/20081; G06T 2207/30048; G06T 2207/30104

(57) ABSTRACT

A method and system for myocardial information determination may be provided. Medical images of the heart of a target subject may be obtained. The medical images may be collected by performing a medical scan on the target subject when the target subject is injected with a first tracer. A target model that has been trained using training samples may be obtained. Each training sample includes label information collected using a second tracer. Blood flow information and/or cardiac function information of the heart of the target subject may be determined based on the medical images and the target model.

### CROSS-REFERENCE TO RELATED APPLICATIONS

This application is a continuation of International Application No. PCT/CN2023/139899, filed on Dec. 19, 2023, which claims priority to Chinese Patent Application No. 202211640229.2 filed on Dec. 20, 2022, the entire contents of which are hereby incorporated by reference.

### TECHNICAL FIELD

The present disclosure generally relates to cardiac analysis, and in particular, to systems and methods for myocardial information determination based on positron emission tomography (PET) imaging.

### BACKGROUND

PET imaging has been widely used in clinical examination and disease diagnosis in recent years. For example, PET data of the heart of a subject is obtained by performing a PET imaging on the heart of the subject. Further, myocardial information (e.g., blood flow information, cardiac function information, myocardial metabolic information, etc.) of the subject is determined based on the PET data of the heart of the subject. Myocardial information is vital for cardiac disease diagnosis.

### SUMMARY

According to an aspect of the present disclosure, a system for myocardial information determination may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain medical images of the heart of a target subject. The medical images may be collected by performing a medical scan on the target subject when the target subject is injected with a first tracer. The system may also obtain a target model that has been trained using training samples, each of which includes label information collected using a second tracer. Further, the system may determine blood flow information and/or cardiac function information of the heart of the target subject based on the medical images and the target model.

In some embodiments, the first tracer may include fludeoxyglucose (FDG) or acetic acid. In some embodiments, the second tracer may include 13N-ammonia (NH3) or Rubidium 82 (Rb82).

In some embodiments, when the target model is used to determine the blood flow information, the medical images may include PET image frames that are consecutively acquired in the medical scan. In some embodiments, a model input of the target model may include segmentation images of the heart by segmenting the heart from the PET image frames or time-activity curves of physical points at the heart.

In some embodiments, when the target model is used to determine the cardiac function information, the medical images may include gated images generated based on a heartbeat signal of the heart collected in the medical scan. In some embodiments, a model input of the target model may include segmentation images of the heart by segmenting the heart from the gated images.

In some embodiments, each training sample of the target model may further include a training input and the training input may be determined by performing the following operations. The system may obtain first sample medical images and second sample medical images of a sample subject. The first sample medical images may be collected by performing a first sample medical scan on the sample subject when the sample subject is injected with the first tracer, and the second sample medical images may be collected by performing a second sample medical scan on the sample subject when the sample subject is injected with the second tracer. The system may determine the label information based on the second sample medical images. Further, the system may determine the training input based on the first sample medical images.

In some embodiments, to determine blood flow information and/or cardiac function information of the heart of the target subject based on the medical images and the target model, the system may perform the following operations. For each of the medical images, the system may generate a pseudo image corresponding to the second tracer by processing the medical image using an image transformation model. The image transformation model may be a trained machine learning model. The system may also determine a model input based on the pseudo image of each medical image. Further, the system may determine the blood flow information and/or the cardiac function information by inputting the model input into the target model. In some embodiments, the training input may be determined by performing the following operations. The system may obtain second sample medical images of a sample subject. The second sample medical images may be collected by performing a second sample medical scan on the sample subject when the sample subject is injected with the second tracer. The system may determine the training input and the label information based on the second sample medical images.

In some embodiments, to determine blood flow information of the heart of the target subject based on the medical images and the target model, the system may perform the following operations. The system may divide the PET image frames into a first subset corresponding to a first stage of the medical scan and a second subset corresponding to a second stage of the medical scan, the first tracer being more concentrated in the blood pool of the heart than the myocardium of the heart in the first stage, the first tracer being more concentrated in the myocardium than the blood pool in the second stage. The system may also determine a first portion of the blood flow information by processing the first subset using at least one pharmacokinetic model. Further, the system may determine a second portion of the blood flow information by processing the second subset using the target model.

In some embodiments, the gated images may include a first group of gated images corresponding to a first stage of the medical scan and a second group of gated images corresponding to a second stage of the medical scan. The first tracer may be more concentrated in the blood pool of the heart than the myocardium of the heart in the first stage, and the first tracer may be more concentrated in the myocardium than the blood pool in the second stage. To determine the cardiac function information of the heart of the target subject based on the medical images and the target model, the system may perform the following operations. For each of one or more candidate gated images of the first group of gated images, the system may determine a left ventricular volume of the target subject corresponding to the candidate gated image. The system may determine a first portion of the cardiac function information based on the one or more left ventricular volumes corresponding to one or more candidate gated images. Further, the system may determine a second portion of the cardiac function information by processing the second group of gated images using the target model.

In some embodiments, the label information may include label blood flow information. The blood flow information may be determined based on the medical images and the target model. The cardiac function information may be determined based on the medical images using another target model that has been trained using training samples, each of which including label cardiac function information collected using the second tracer.

In some embodiments, the label information may include label cardiac function information. The cardiac function information may be determined based on the medical images and the target model. The blood flow information may be determined based on the medical images using another target model that has been trained using training samples, each of which includes label blood flow information collected using the second tracer.

In some embodiments, the label information may include label blood flow information and label cardiac function information. The target model may be a multitask model, and both the blood flow information and the cardiac function information may be determined based on the medical images and the multitask model.

In some embodiments, the system may further determine myocardial metabolic information of the heart by processing the medical images using a second pharmacokinetic model.

According to an aspect of the present disclosure, a system for myocardial information determination may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain PET data of the heart of a target subject. The PET data may be collected by performing a medical scan on the target subject when the target subject is injected with a tracer. Further, the system may determine at least two of blood flow information, cardiac function information, or myocardial metabolic information of the heart of the target subject based on the PET data of the heart of the target subject.

In some embodiments, a first portion of the PET data for determining the blood flow information may be collected in a first time period from when the tracer is injected.

In some embodiments, the first time period for collecting the first portion of the PET data or a second time period for collecting a second portion of the PET data may be smaller than a third time period for collecting a third portion of the PET data. The first portion of the PET data may be used to determine the blood flow information, the second portion of the PET data may be used to determine the cardiac function information, and the third portion of the PET data may be used to determine the myocardial metabolic information.

In some embodiments, to determine at least two of blood flow information, cardiac function information, or myocardial metabolic information of the heart of the target subject based on the PET data of the heart of the target subject, the system may perform the following operations. The system may generate medical images of the heart of the target subject based on the PET data of the heart of the target subject. The system may obtain the target model that has been trained using training samples, each of which includes label information collected using the second tracer. Further, the system may determine the blood flow information and/or the cardiac function information of the heart of the target subject based on the medical images and the target model.

According to yet another aspect of the present disclosure, a method for myocardial information determination may be provided. The method may include obtaining medical images of the heart of a target subject. The medical images may be collected by performing a medical scan on the target subject when the target subject is injected with a first tracer. The method may also include obtaining a target model that has been trained using training samples, each of which includes label information collected using a second tracer. The method may further include determining blood flow information and/or cardiac function information of the heart of the target subject based on the medical images and the target model.

According to yet another aspect of the present disclosure, a method for myocardial information determination may be provided. The method may include obtaining PET data of the heart of a target subject. The PET data may be collected by performing a medical scan on the target subject when the target subject is injected with a tracer. The method may further include determining at least two of blood flow information, cardiac function information, or myocardial metabolic information of the heart of the target subject based on the PET data of the heart of the target subject.

According to yet another aspect of the present disclosure, a system for myocardial information determination may be provided. The system may include an obtaining module and a determination module. The obtaining module may be configured to obtain medical images of the heart of a target subject. The medical images may be collected by performing a medical scan on the target subject when the target subject is injected with a first tracer. The obtaining module may be also configured to obtain a target model that has been trained using training samples, each of which includes label information collected using a second tracer. The determination module may be configured to determine blood flow information and/or cardiac function information of the heart of the target subject based on the medical images and the target model.

According to yet another aspect of the present disclosure, a system for myocardial information determination may be provided. The system may include an obtaining module and a determination module. The obtaining module may be configured to obtain PET data of the heart of a target subject. The PET data may be collected by performing a medical scan on the target subject when the target subject is injected with a tracer. The determination module may be configured to determine at least two of blood flow information, cardiac function information, or myocardial metabolic information of the heart of the target subject based on the PET data of the heart of the target subject.

According to yet another aspect of the present disclosure, a non-transitory computer readable medium may be provided. The non-transitory computer readable medium may include at least one set of instructions for medical imaging. When executed by one or more processors of a computing device, the at least one set of instructions may cause the computing device to perform a method. The method may include obtaining medical images of the heart of a target subject. The medical images may be collected by performing a medical scan on the target subject when the target subject is injected with a first tracer. The method may also include obtaining a target model that has been trained using training samples, each of which includes label information collected using a second tracer. The method may further include determining blood flow information and/or cardiac function information of the heart of the target subject based on the medical images and the target model.

According to yet another aspect of the present disclosure, a non-transitory computer readable medium may be provided. The non-transitory computer readable medium may include at least one set of instructions for medical imaging. When executed by one or more processors of a computing device, the at least one set of instructions may cause the computing device to perform a method. The method may include obtaining PET data of the heart of a target subject. The PET data may be collected by performing a medical scan on the target subject when the target subject is injected with a tracer. The method may further include determining at least two of blood flow information, cardiac function information, or myocardial metabolic information of the heart of the target subject based on the PET data of the heart of the target subject.

According to yet another aspect of the present disclosure, a device for myocardial information determination may be provided. The device may include at least one processor and at least one storage device for storing a set of instructions. When the set of instructions may be executed by the at least one processor, the device performs the methods for myocardial information determination.

Additional features may be set forth in part in the description which follows, and in part may become apparent to those skilled in the art upon examination of the following and the accompanying drawings or may be learned by the production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary PET system according to some embodiments of the present disclosure;

FIG. 2 is a schematic diagram illustrating exemplary hardware and/or software components of an exemplary computing device according to some embodiments of the present disclosure;

FIG. 3 is a block diagram illustrating exemplary processing device according to some embodiments of the present disclosure;

FIG. 4 is a flowchart illustrating an exemplary process for myocardial information determination according to some embodiments of the present disclosure;

FIG. 5 is a schematic diagram illustrating training and application process of an exemplary target model A according to some embodiments of the present disclosure;

FIG. 6 is a schematic diagram illustrating training and application process of an exemplary target model B according to some embodiments of the present disclosure;

FIG. 7 is a schematic diagram illustrating an exemplary process for determining blood flow information and cardiac function information via different target models according to some embodiments of the present disclosure;

FIG. 8 is a schematic diagram illustrating an exemplary process for determining blood flow information and cardiac function information via a multitask model according to some embodiments of the present disclosure;

FIG. 9 is a schematic diagram illustrating exemplary cardiac function information of a subject according to some embodiments of the present disclosure; and

FIG. 10 is a flowchart illustrating an exemplary process for myocardial information determination according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

An anatomical structure shown in an image of a subject (e.g., a patient) may correspond to an actual anatomical structure existing in or on the subject's body. The term “object” and “subject” in the present disclosure are used interchangeably to refer to a biological object (e.g., a patient, an animal) or a non-biological object (e.g., a phantom). In some embodiments, the object may include a specific part, organ, and/or tissue of the object. For example, the object may include the head, the bladder, the brain, the neck, the torso, a shoulder, an arm, the thorax, the heart, the stomach, a blood vessel, soft tissue, a knee, a foot, or the like, or any combination thereof, of a patient.

These and other features, and characteristics of the present disclosure, as well as the methods of operation and functions of the related elements of structure and the combination of parts and economics of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Conventionally, two PET scans with two different tracers need to be performed on a subject to obtain accurate and comprehensive myocardial information of the subject. As used herein, comprehensive myocardial information includes the blood flow information, the cardiac function information, and the myocardial metabolic information. The two PET scans normally include a first PET scan using FDG and a second PET scan using 13N-ammonia (NH3) or Rubidium 82 (Rb82). Since FDG participates in myocardial metabolism while NH3 and Rb82 do not participate in myocardial metabolism, myocardial metabolic information is determined based on PET data collected in the first PET scan. Since metabolites of the FDG stay in the myocardium, blood flow information and cardiac function information are determined based on PET data collected in the second scan. The conventional approach is inefficient and unfriendly to the subject. Moreover, the NH3 and Rb82 have a short half-life and are difficult to obtain.

An aspect of the present disclosure provides systems and methods for myocardial information determination based on PET imaging. The systems may obtain medical images of the heart of a target subject. The medical images may be collected by performing a medical scan on the target subject when the target subject is injected with a first tracer. The systems may also obtain a target model that has been trained using training samples. Each training sample may include label information collected using a second tracer. Further, the systems may determine blood flow information and/or cardiac function information of the heart of the target subject based on the medical images and the target model. In some embodiments, the systems may further determine myocardial metabolic information of the heart by processing the medical images using a second pharmacokinetic model. Compared with the conventional approach, the systems and methods of the present discloser may obtain accurate and comprehensive myocardial information with PET data collected by only one medical scan, which is more efficient and friendly to the subject.

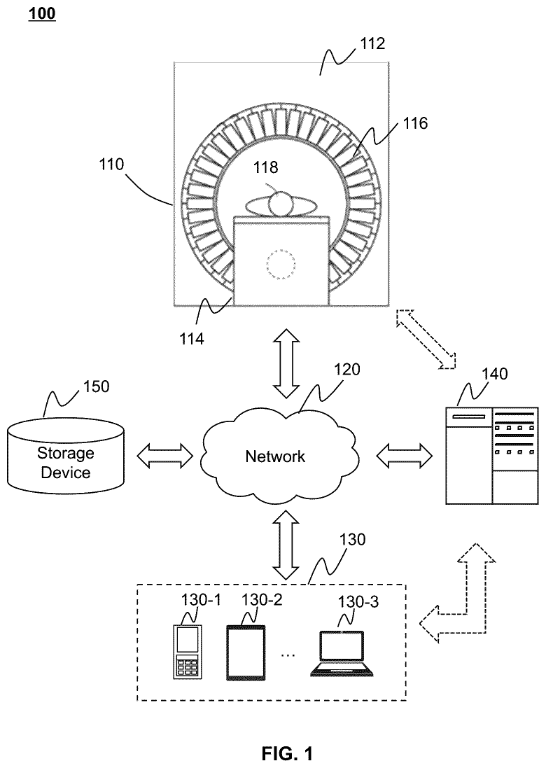

FIG. 1 is a schematic diagram illustrating an exemplary PET system according to some embodiments of the present disclosure. In some embodiments, as illustrated in FIG. 1, the PET system 100 may include a PET scanner 110, a network 120, a terminal device 130, a processing device 140, and a storage device 150. The components of the PET system 100 may be connected in various manners.

The PET scanner 110 may be configured to acquire scan data relating to an object. For example, the PET scanner 110 may scan the object or a portion thereof that is located within its detection region and generate the scan data relating to the object or the portion thereof.

In some embodiments, the PET scanner 110 may include a gantry 112, a couch 114, and a detector 116. The gantry 112 may support the detector 116. The couch 114 may be used to support an object 118 (or referred to as a subject) to be scanned. The detector 116 may include a plurality of detector rings arranged along an axial direction (e.g., Z-axis direction in FIG. 1) of the gantry 112. In some embodiments, a detector ring may include a plurality of detector units arranged along the circumference of the detector ring. In some embodiments, the detector 116 may include a scintillation detector (e.g., a cesium iodide detector), a gas detector, or the like, or any combination thereof.

In some embodiments, before a PET scanning, the object 118 may be injected with a tracer. The tracer may refer to a radioactive substance that decays and emits positrons. In some embodiments, the tracer may be radioactively marked radiopharmaceutical, which is a drug having radioactivity and is administered to the object 118. For example, the tracer may include fluorine-18 (¹⁸F) fluorodeoxyglucose (FDG), etc. During the scanning, pairs of photons (e.g., gamma photons) may result from the annihilation of positrons originating from the tracer in the object 118. A pair of photons may travel in opposite directions. At least a part of the pairs of photons may be detected and/or registered by the detector units in the detector 116. A coincidence event may be recorded when a pair of photons generated by the positron-electron annihilation are detected within a coincidence time window (e.g., within 6 to 12 nanoseconds). The coincidence event may be assumed to occur along a line connecting a pair of detector units, and the line may be called as a “line of response” (LOR). The detector 116 may obtain counts of coincidence events based on the LORs for detected coincidence events and time points at which the coincidence events occurred.

In some embodiments, the PET scanner 110 may also be a multi-modality scanner, for example, a positron emission tomography-computed tomography (PET-CT) scanner, etc.

The network 120 may facilitate exchange of information and/or data. In some embodiments, one or more components (e.g., the PET scanner 110, the terminal device 130, the processing device 140, the storage device 150) of the PET system 100 may send information and/or data to other component(s) of the PET system 100 via the network 120. For example, the processing device 140 may obtain, via the network 120, scan data relating to the object 118 or a portion thereof from the PET scanner 110. In some embodiments, the network 120 may be any type of wired or wireless network, or a combination thereof.

The terminal device 130 may include a mobile device 130-1, a tablet computer 130-2, a laptop computer 130-3, or the like, or any combination thereof. In some embodiments, the terminal device 130 may remotely operate the PET scanner 110. In some embodiments, the terminal device 130 may operate the PET scanner 110 via a wireless connection. In some embodiments, the terminal device 130 may receive information and/or instructions inputted by a user, and send the received information and/or instructions to the PET scanner 110 or the processing device 140 via the network 120. In some embodiments, the terminal device 130 may receive data and/or information from the processing device 140. In some embodiments, the terminal device 130 may be part of the processing device 140. In some embodiments, the terminal device 130 may be omitted.

The processing device 140 may process data obtained from the PET scanner 110, the terminal device 130, the storage device 150, or other components of the PET system 100. For example, the processing device 140 may obtain scan data (e.g., medical images) of an object (e.g., a human body) from the PET scanner 110 or the storage device 150, and determine myocardial information of the heart of the object based on the scan data of the object by performing the process 400.

In some embodiments, the processing device 140 (e.g., one or more modules illustrated in FIG. 3) may execute instructions and may accordingly be directed to perform one or more processes (e.g., processes 400 and 1000) described in the present disclosure. For example, each of the one or more processes may be stored in a storage device (e.g., the storage device 150) as a form of instructions, and invoked and/or executed by the processing device 140.

In some embodiments, the processing device 140 may be a single server or a server group. The server group may be centralized or distributed. In some embodiments, the processing device 140 may be local or remote. Merely for illustration, only one processing device 140 is described in the medical system 100. However, it should be noted that the medical system 100 in the present disclosure may also include multiple processing devices. Thus, operations and/or method steps that are performed by one processing device 140 as described in the present disclosure may also be jointly or separately performed by the multiple processing devices. For example, if in the present disclosure the processing device 140 of the medical system 100 executes both process A and process B, it should be understood that the process A and the process B may also be performed by two or more different processing devices jointly or separately in the medical system 100 (e.g., a first processing device executes process A and a second processing device executes process B, or the first and second processing devices jointly execute processes A and B).

The storage device 150 may store data, instructions, and/or any other information. In some embodiments, the storage device 150 may store data obtained from the processing device 140, the terminal device 130, and/or the PET scanner 110. For example, the storage device 150 may store scan data collected by the PET scanner 110. As another example, the storage device 150 may store the myocardial information of the object. In some embodiments, the storage device 150 may store data and/or instructions that the processing device 140 may execute or use to perform exemplary methods described in the present disclosure.

It should be noted that the above description of the PET system 100 is merely provided for the purposes of illustration, and not intended to limit the scope of the present disclosure. For persons having ordinary skills in the art, multiple variations and modifications may be made under the teachings of the present disclosure. For example, the PET system 100 may include one or more additional components and/or one or more components of the PET system 100 described above may be omitted. Additionally or alternatively, two or more components of the PET system 100 may be integrated into a single component. A component of the PET system 100 may be implemented on two or more sub-components.

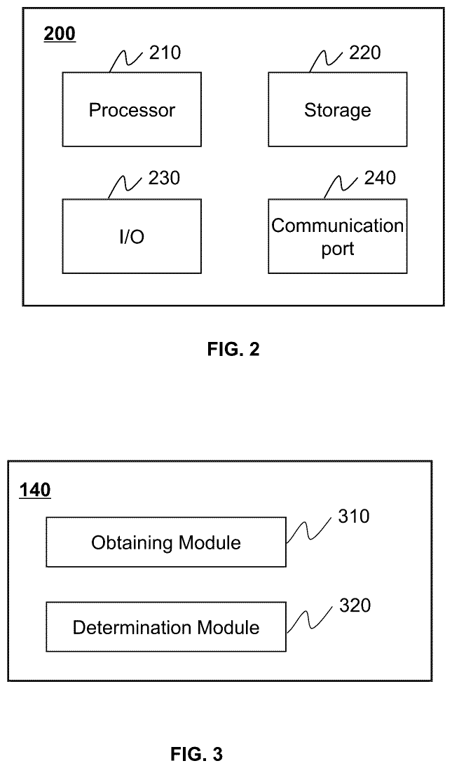

FIG. 2 is a schematic diagram illustrating exemplary hardware and/or software components of an exemplary computing device according to some embodiments of the present disclosure. In some embodiments, the processing device 140 may be implemented on the computing device 200. As illustrated in FIG. 2, the computing device 200 may include a processor 210, a storage 220, an input/output (I/O) 230, and a communication port 240.

The processor 210 may execute computer instructions (program code) and perform functions of the processing device 140 in accordance with techniques described herein. The computer instructions may include routines, programs, objects, components, signals, data structures, procedures, modules, and functions, which perform particular functions described herein. Merely for illustration purposes, only one processor is described in the computing device 200. However, it should be noted that the computing device 200 in the present disclosure may also include multiple processors, and thus operations of a method that are performed by one processor as described in the present disclosure may also be jointly or separately performed by the multiple processors.

The storage 220 may store data/information obtained from the PET scanner 110, the terminal device 130, the storage device 150, or any other component of the PET system 100. In some embodiments, the storage 220 may include a mass storage device, a removable storage device, a volatile read-and-write memory, a read-only memory (ROM), or the like, or any combination thereof. In some embodiments, the storage 220 may store one or more programs and/or instructions to perform exemplary methods described in the present disclosure.

The I/O 230 may input or output signals, data, or information. In some embodiments, the I/O 230 may enable user interaction with the processing device 140. In some embodiments, the I/O 230 may include an input device and an output device.

The communication port 240 may be connected to a network (e.g., the network 120) to facilitate data communications. The communication port 240 may establish connections between the processing device 140 and the PET scanner 110, the terminal device 130, or the storage device 150. The connection may be a wired connection, a wireless connection, or combination of both that enables data transmission and reception.

It should be noted that the above description of the computing device 200 is merely provided for the purposes of illustration, and not intended to limit the scope of the present disclosure. For persons having ordinary skills in the art, multiple variations and modifications may be made under the teachings of the present disclosure.

FIG. 3 is a block diagram illustrating exemplary processing device according to some embodiments of the present disclosure. As shown in FIG. 3, the processing device 140 may include an obtaining module 310 and a determination module 320. In some embodiments, as described in FIG. 1, the PET system 100 in the present disclosure may also include multiple processing devices, and the obtaining module 310, the determination module 320 may be components of different processing devices.

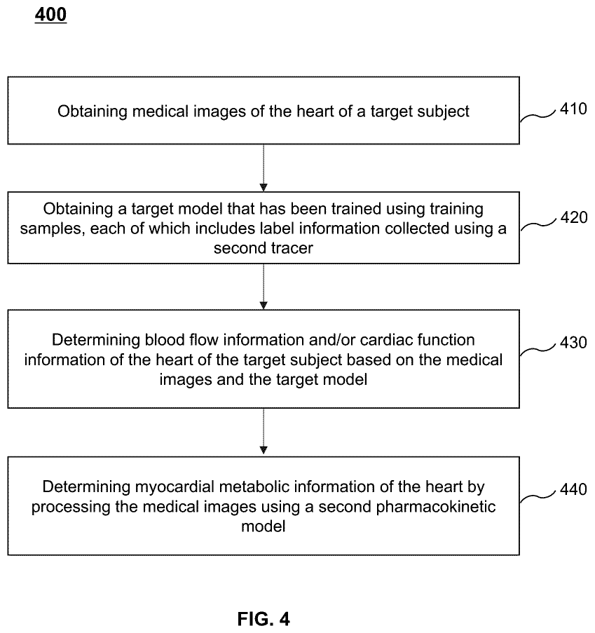

The obtaining module 310 may be configured to obtain information relating to the PET system 100. For example, the obtaining module 310 may obtain medical images of the heart of a target subject. The medical images may be collected by performing a medical scan on the target subject when the target subject is injected with a first tracer. More descriptions regarding the obtaining of the medical images of the heart of the target subject may be found elsewhere in the present disclosure. Sec, e.g., operation 410 in FIG. 4, and relevant descriptions thereof. As another example, the obtaining module may obtain PET data of the heart of a target subject. The PET data may be collected by performing a medical scan on the target subject when the target subject is injected with a tracer. More descriptions regarding the obtaining of the PET data may be found elsewhere in the present disclosure. See, e.g., operation 1010 in FIG. 10, and relevant descriptions thereof. As still another example, the obtaining module may obtain a target model that has been trained using training samples, each of which includes label information collected using a second tracer. More descriptions regarding the obtaining of the target model may be found elsewhere in the present disclosure. See, e.g., operation 420 in FIG. 4, and relevant descriptions thereof.

In some embodiments, the determination module may be configured to determine blood flow information and/or cardiac function information of the heart of the target subject based on the medical images and the target model. More descriptions regarding the determination of the blood flow information and/or the cardiac function information may be found elsewhere in the present disclosure. See, e.g., operation 430 in FIG. 4, and relevant descriptions thereof.

In some embodiments, the determination module may be also configured to determine myocardial metabolic information of the heart by processing the medical images using a second pharmacokinetic model. More descriptions regarding the determination of the myocardial metabolic information may be found elsewhere in the present disclosure. See, e.g., operation 440 in FIG. 4, and relevant descriptions thereof.

In some embodiments, the determination module may be configured to determine at least two of blood flow information, cardiac function information, or myocardial metabolic information of the heart of the target subject based on the PET data of the heart of the target subject. More descriptions regarding the determining at least two of the blood flow information, the cardiac function information, or the myocardial metabolic information may be found elsewhere in the present disclosure. See, e.g., operation 1020 in FIG. 10, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the obtaining module 310 may be divided into two units configured to acquire different data. In some embodiments, the processing device 140 may include one or more additional modules, such as a storage module (not shown) for storing data.

FIG. 4 is a flowchart illustrating an exemplary process for myocardial information determination according to some embodiments of the present disclosure.

In 410, the processing device 140 (e.g., the obtaining module 310) may obtain medical images of the heart of a target subject. The medical images may be collected by performing a medical scan on the target subject when the target subject is injected with a first tracer.

In some embodiments, the first tracer may be metabolized in the myocardium of the heart. Thus, the first tracer may be used to determine myocardial metabolic information. In some embodiments, since metabolites of the first tracer metabolized in the myocardium of the heart can not be absorbed by the myocardium (i.e., remain in the myocardium), blood flow information and cardiac function information of the heart determined based on PET data collected by the first tracer have a relatively low accuracy. In some embodiments, the first tracer may have a relatively long half-life and be difficult to obtain.

In some embodiments, the first tracer may include FDG (e.g., 18F-FDG), acetic acid, or the like.

In some embodiments, PET data may be collected by performing the medical scan on a region of interest (ROI) of the target subject via the PET scanner 110 over a scan time period. The target subject may include a patient, an animal, a phantom, or a portion thereof. The ROI may include the heart (or a portion thereof, e.g., the left ventricular) of the target subject.

In some embodiments, the processing device 140 may obtain the PET data from one or more components (e.g., the PET scanner 110, the storage device 150) of the PET system 100 or an external source via a network (e.g., the network 120), and generate the medical images based on the PET data. Alternatively, the processing device 140 may directly obtain the medical images from another component (e.g., the PET scanner, the storage device 150).

In some embodiments, the processing device 140 may divide the PET data into sets of PET data and reconstruct the medical images based on the sets of PET data using a reconstruction algorithm. Exemplary reconstruction algorithms may include a maximum-likelihood reconstruction of attenuation and activity (MLAA) algorithm, an iterative reconstruction algorithm (e.g., a statistical reconstruction algorithm), a Fourier slice theorem algorithm, a filtered back projection (FBP) algorithm, a compressed sensing (CS) algorithm, a fan-beam reconstruction algorithm, a maximum likelihood expectation maximization (MLEM) algorithm, an ordered subset expectation maximization (OSEM) algorithm, a maximum a posterior (MAP) algorithm, an analytic reconstruction algorithm, or the like, or any combination thereof.

In some embodiments, the processing device 140 may divide the scan time period into sub-time periods in sequence. Further, the processing device 140 may divide the PET data into the sets of PET data according to the sub-time periods. Specifically, the processing device 140 may designate PET data collected in each of the sub-time periods as one set of the sets of PET data. For example, the scan time period may be 10 minutes, and the processing device 140 may divide the scan time period into 600 sub-time periods each of which is 1 second. Then, the processing device 140 may designate PET data collected in each second as a set of PET data. In such cases, the medical images reconstructed based on the sets of PET data may include PET image frames that are consecutively acquired in the medical scan.

In some embodiments, the processing device 140 may obtain a heartbeat signal of the heart collected in the medical scan. The processing device 140 may divide the heart signal into multiple parts each of which corresponds to one of multiple heartbeat phases. In some embodiments, the heart signal may be divided according to the amplitude of the heart signal. For example, a cycle of the heart signal may be divided based on the amplitude of the heart signal. The amplitude of the heart signal is evenly segmented into n parts (e.g., from the maximum amplitude to the minimum amplitude), thereby generating n portions of the heart signal corresponding to n heartbeat phases. In some embodiments, the heart signal may be divided into N parts based on the time, and the N parts may correspond to N heartbeat phases. For example, if a cycle of the heart signal lasts 5 seconds, a cycle of the heart signal may be divided according to a unit interval (e.g., 0.5 seconds, or 1 second), and this cycle of the heart signal may be divided into N heartbeat phases (e.g., 5/0.5 heartbeat phases, or 5/1 heartbeat phases).

Further, the processing device 140 may gate the PET data into multiple sets of gated PET data based on the multiple heartbeat phases and designate the multiple sets of gated PET data as the sets of PET data. Each set of gated PET data may correspond to one of the multiple heartbeat phases. For example, the heart signal may include N heartbeat phases, and the processing device 140 may gate the PET data into N sets of gated PET data each of which corresponds to one of the N heartbeat phases. A set of gated PET data corresponding to a specific heartbeat phase is collected when the target subject is in the specific heartbeat phase. In such cases, the medical images reconstructed based on the sets of PET data may include gated images corresponding to the multiple heartbeat phases.

In 420, the processing device 140 (e.g., the obtaining module 310) may obtain a target model that has been trained using training samples, each of which includes label information collected using a second tracer.

The target model may be a trained model (e.g., a machine learning model) for determining blood flow information and/or cardiac function information of the heart. In some embodiments, the target model may be only used to determine blood flow information of the heart. In some embodiments, the target model may be only used to determine cardiac function information of the heart. In some embodiments, the target model may be used to determine both blood flow information and cardiac function information of the heart of the target subject. In some embodiments, the target model may include a deep learning model, such as a deep neural network (DNN) model, a convolutional neural network (CNN) model, a recurrent neural network (RNN) model, a feature pyramid network (FPN) model, etc. Exemplary CNN models may include a V-Net model, a U-Net model, a Link-Net model, or the like, or any combination thereof.

In some embodiments, the processing device 140 may obtain the target model from one or more components (e.g., the storage device 150, the storage 210) of the PET system 100 or an external source via a network (e.g., the network 120). For example, the target model may be previously trained by a computing device (e.g., the processing device 140 or other processing devices), and stored in a storage device (e.g., the storage device 150, the storage 210) of the PET system 100. The processing device 140 may access the storage device and retrieve the target model.

In some embodiments, different from the first tracer, the second tracer is not metabolized in the myocardium of heart, and PET data collected by the second tracer is more suitable for determining the blood flow information and/or the cardiac function information. Exemplary second tracers may include 13N-ammonia (NH3), Rubidium 82 (Rb82), etc.

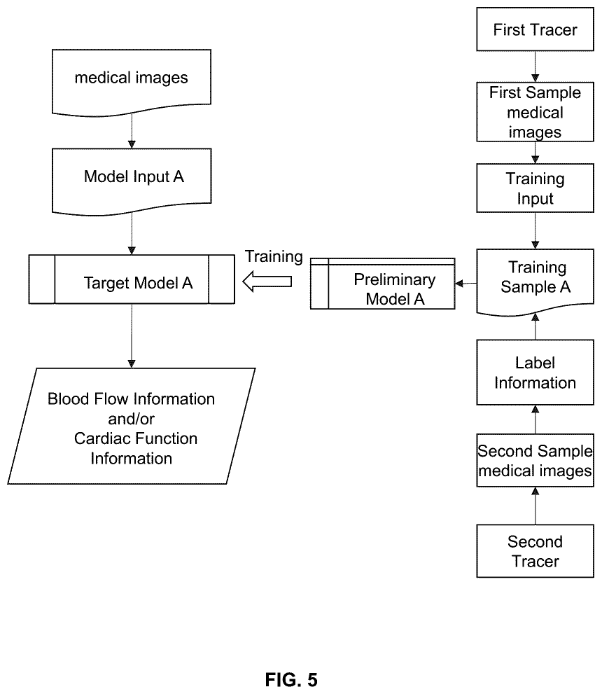

In some embodiments, the target model may be a target model A whose training samples are collected using the first tracer and the second tracer. For example, FIG. 5 is a schematic diagram illustrating training and application process of an exemplary target model A according to some embodiments of the present disclosure. As shown in FIG. 5, the target model A may be obtained by training a preliminary model A using multiple training samples A. Each training sample A may include a training input and label information. In some embodiments, the processing device 140 may obtain first sample medical images and second sample medical images of a sample subject. The first sample medical images may be collected by performing a first sample medical scan on the sample subject when the sample subject is injected with the first tracer. The second sample medical images may be collected by performing a second sample medical scan on the sample subject when the sample subject is injected with the second tracer. In some embodiments, the first sample medical images and the second sample medical images may be generated in a similar manner as how the medical images described in operation 410 are generated, and the descriptions thereof are not repeated here.

The processing device 140 may determine the training input based on the first sample medical images. In some embodiments, when the target model A is used to determine the blood flow information, the first sample medical images may include sample PET image frames that are consecutively acquired in the first sample medical scan. Specifically, for each training sample A, the processing device 140 may generate a sample segmentation image of the heart of the sample subject by segmenting the heart from each sample PET image frame in the training sample A. Further, the processing device 140 may determine the training input based on the sample segmentation images. In some embodiments, the training input may include the sample segmentation images. In some embodiments, the training inputs may include sample time-activity curves of physical points at the heart of the sample subject determined based on the sample segmentation images.

In some embodiments, when the target model A is used to determine the cardiac function information, the first medical images may include sample gated images generated based on a heartbeat signal of the heart of the sample subject collected in the first sample medical scan. The training input may include sample segmentation images of the heart of the sample subject by segmenting the heart from the sample gated images.

In some embodiments, the processing device 140 may determine the training input in a similar manner as how the model input is determined as described in operation 430.

If the target model A is only used to determine blood flow information, the label information of a training sample A may include label blood flow information of the heart of the sample subject in the training sample A. If the target model A is only used to determine cardiac function information, the label information of a training sample A may include label cardiac function information of the heart of the sample subject in the training sample A. If the target model A is used to determine both blood flow information and cardiac function information, the label information of a training sample A may include label blood flow information and label cardiac function information of the heart of the sample subject in the training sample A. In some embodiments, the processing device 140 may determine the label information based on the second sample medical images. For example, for each training sample A, the processing device 140 may determine the label blood flow information of the training sample by processing the second sample medical images or sample segmentation images of the heart of the sample subject segmented from the second sample medical images using a pharmacokinetic algorithm (e.g., one or more pharmacokinetic models) or a principal component analysis algorithm. As another example, for each training sample A, the processing device 140 may determine the label cardiac function information of the training sample by determining one or more left ventricular volumes of the sample subject each of which corresponds to one of the second sample medical images.

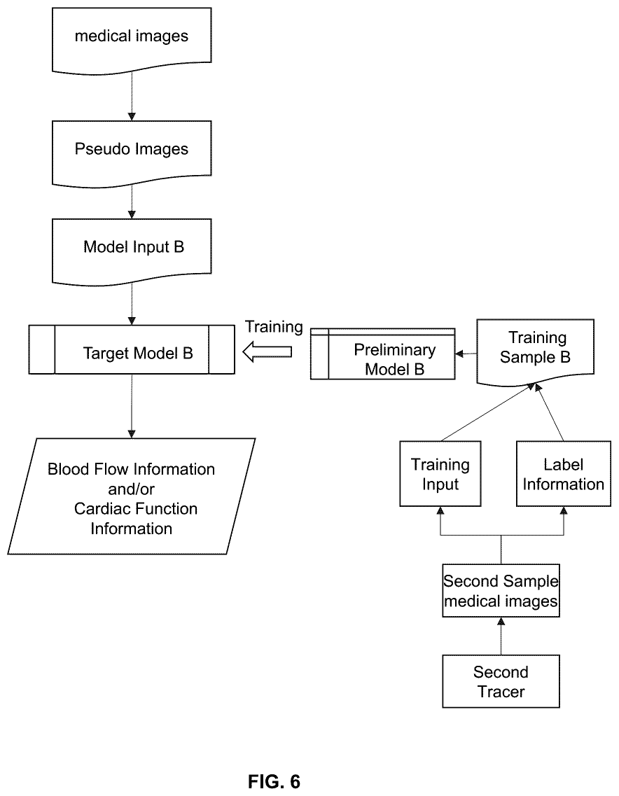

In some embodiments, the target model may be a target model B whose training samples are collected only using the second tracer. For example, FIG. 6 is a schematic diagram illustrating training and application process of an exemplary target model B according to some embodiments of the present disclosure. As shown in FIG. 6, the target model B may be obtained by training a preliminary model B using multiple training samples B. Each training sample B may include a training input and label information. In some embodiments, the processing device 140 may obtain second sample medical images of a sample subject. The second sample medical images may be collected by performing a second sample medical scan on the sample subject when the sample subject is injected with the second tracer. Further, the processing device 140 may determine the training input and the label information based on the second sample medical images. In some embodiments, the processing device 140 may determine the training input of a training sample B based on the second sample medical images in a similar manner as how the training input of a training sample A is determined based on the first sample medical images. In some embodiments, the processing device 140 may determine the label information of a training sample B in a similar manner as how the label information of a training sample A is determined.

Since the second tracer is more suitable for determining blood flow information and cardiac function information, the label information collected using the second tracer may have a relatively large accuracy, the target model trained using the label information may can determine accurate blood flow information and/or cardiac function information.

In 430, the processing device 140 (e.g., the determination module 320) may determine blood flow information and/or cardiac function information of the heart of the target subject based on the medical images and the target model.

The blood flow information may include blood flow parameters in vessels of the heart of the target subject. Exemplary blood flow information may include a blood flow velocity, a total blood flow volume, a region total activity of blood flow, a blood flow resistance, a blood pressure, a shear stress, a disturbance flow, etc. The blood flow velocity refers to a linear moving velocity of a particle in the blood. The total blood flow volume refers to a blood volume flowing through a certain section of the vessels per unit time. The region total activity of the blood flow refers to an activity of the blood in a unit region of the vessels.

The cardiac function information is used to indicate the ability of the heart tissue to contract and pump blood. The cardiac function information may include a cardiac output, an end-systolic volume (ESV), an end-diastolic volume (EDV), an ejection fraction (EF), etc.

In some embodiments, as shown in FIG. 5, the processing device 140 may determine a model input A based on the medical images, and determine the blood flow information and/or the cardiac function information by inputting the model input A into the target model A.

In some embodiments, when the target model A is used to determine the blood flow information, the medical images include the PET image frames that are consecutively acquired in the medical scan described in operation 410. The model input A of the target model A may include the PET image frames, the segmentation images (also referred to as first segmentation images) of the heart by segmenting the heart from the PET image frames, time-activity curves (TACs) (also referred to as first TACs) of physical points at the heart, or the like, or any combination thereof. Specifically, the processing device 140 may generate the first segmentation images of the heart by segmenting the heart from the PET image frames. In some embodiments, the heart of target subject may be segmented from the PET image frames manually by a user (e.g., a doctor, an imaging specialist, a technician) by, for example, drawing bounding boxes on the PET image frames displayed on a user interface. Alternatively, the PET image frames may be segmented by the processing device 140 automatically according to an image analysis algorithm (e.g., an image segmentation algorithm). Exemplary image segmentation algorithm may include a thresholding segmentation algorithm, a compression-based algorithm, an edge detection algorithm, a machine learning-based segmentation algorithm, a 17-segmentation algorithm, a pixel segmentation algorithm, or the like, or any combination thereof. According to the 17-segmentation algorithm, 17 segments of the heart, including basal segments of the anterior septum, basal segments of the anterior wall, and basal segments of the lateral wall, etc., may be segmented from the PET image frames. The pixel segmentation algorithm refers to an algorithm that segments the heart from an image according to relationships between pixels in the image (e.g., the relationship between the blood pool edge pixels and the myocardial edge pixels). In some embodiments, the processing device 140 may perform a preprocessing (e.g., a correction processing) on the segmentation images of the heart. For example, the processing device 140 may perform a correction processing on the segmentation images of the heart by removing pixels that do not satisfy the requirements from the segmentation images.

Further, the processing device 140 may determine the model input A based on the first segmentation images. In some embodiments, the model input A may include the first segmentation images. In some embodiments, the model input A may include the first TACs of physical points at the heart. The processing device 140 may determine the first TACs of physical points at the heart based on the first segmentation images. Specifically, for each physical point at the heart, the processing device 140 may determine a pixel value or an activity value corresponding to the physical point in each of the PET image frames. Further, the pixel values or activity values determined based on the pixel values may be arranged in a chronological order, the processing device 140 may generate a first TAC corresponding to the physical point based on the arranged pixel values or the arranged activity values using a fitting algorithm.

In some embodiments, when the target model A is used to determine the cardiac function information, the medical images may include the gated images generated based on the heartbeat signal of the heart collected in the medical scan described in operation 410. The model input A of the target model A may include the gated images, segmentation images (also referred to as second segmentation images) of the heart by segmenting the heart from the gated images. In some embodiments, the second segmentation images may be generated in a similar manner as the generation of the first segmentation images.

In some embodiments, as shown in FIG. 6, the processing device 140 may determine a model input B based on the medical images, and determine the blood flow information and/or the cardiac function information by inputting the model input B into the target model B. Specifically, for each medical image, the processing device 140 may generate a pseudo image corresponding to the second tracer by processing the medical image using an image transformation model. The pseudo images may be similar to medical images collected by performing a PET scan on the target subject when the target subject is injected with the second tracer. The image transformation model may be a trained machine learning model (e.g., a generative adversarial net (GAN) model) for transforming images corresponding to different tracers. Specifically, the medical image may be input into the image transformation model, and the image transformation model may output the pseudo image.

In some embodiments, the processing device 140 may obtain the image transformation model in a similar manner as how the target model is obtained. In some embodiments, the image transformation model may be obtained by training a preliminary model using multiple training samples. Each training sample may include a first sample image and a second sample image of a sample subject. The first sample image may be collected by performing a first sample medical scan on the sample subject when the sample subject is injected with the first tracer. The second sample image may be collected by performing a second sample medical scan on the sample subject when the sample subject is injected with the second tracer. The second sample image can be used as a ground truth (also referred to as a label) for model training.

For example, the preliminary model may be the GAN model. The GAN model may include a generator and a discriminator. The training of the GAN model may include one or more iterations. In the current iteration, the first sample image of the training sample may be input into the generator, the generator may generate a predict pseudo image. Then, the discriminator may generate a discrimination result between the pseudo image and the second sample image. The model parameters of the GAN model may be iteratively updated such that the generator can generate a predicted pseudo image as close as possible to the second sample image (i.e., the true image corresponding to the second tracer) to fool the discriminator, and the discriminator can accurately distinguish between the predicted pseudo image and the second sample image. When a termination condition is satisfied, the iterations will be stopped, and the generator of the GAN model may be designated as the image transformation model.

Further, the processing device 140 may determine the model input B based on the pseudo image of each medical image. In some embodiments, when the target model B is used to determine the blood flow information, the medical images include the PET image frames that are consecutively acquired in the medical scan described in operation 410. The model input B of the target model B may include pseudo images corresponding to the PET image frames, the segmentation images (also referred to as third segmentation images) of the heart by segmenting the heart from the pseudo images, second TACs of physical points at the heart obtained generated based on the third segmentation images, or the like, or any combination thereof. In some embodiments, the processing device 140 may determine the second TACs of physical points at the heart based on the third segmentation images in a similar manner as how the first TACs are determined, and the descriptions thereof are not repeated here.

As described elsewhere in the present disclosure, in some embodiments, the target model (e.g., the target model A, the target model B) may be only used to determine blood flow information of the heart, and such target model may be also referred to as the blood flow determination model. In some embodiments, the target model (e.g., the target model A, the target model B) may be only used to determine cardiac function information of the heart, and such target model may be also referred to as the cardiac function determination model. Accordingly, in some embodiments, the blood flow information and cardiac function information of the heart may be determined via different target models (e.g., the blood flow determination model and the cardiac function determination model).

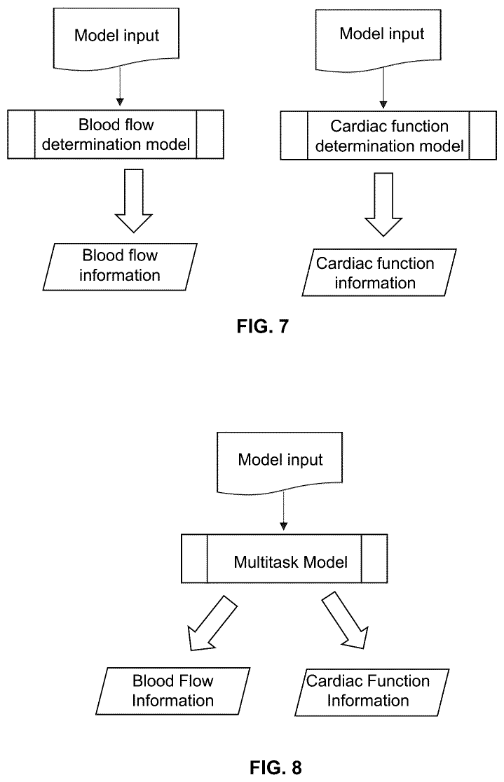

For example, FIG. 7 is a schematic diagram illustrating an exemplary process for determining blood flow information and cardiac function information via different target models according to some embodiments of the present disclosure. As shown in FIG. 7, the model input may be respectively input into the blood flow determination model and the cardiac function determination model. The blood flow determination model may output the blood flow information of the heart, and the cardiac function determination model may output the cardiac function information of the heart.

In some embodiments, if the target model obtained in operation 420 is the blood flow determination model, the label information described in operation 420 may include label blood flow information, and the blood flow information may be determined based on the medical images (e.g., the PET image frames) and the blood flow determination model. After operation 430, the processing device 140 may further determine the cardiac function information based on the medical images (e.g., the gated images) using another target model (i.e., the cardiac function determination model) that has been trained using training samples, each of which including label cardiac function information collected using the second tracer.

In some embodiments, if the target model obtained in operation 420 is the cardiac function determination model, the label information described in operation 420 may include label cardiac function information, and the cardiac function information may be determined based on the medical images (e.g., the gated images) and the cardiac function determination model. After operation 430, the processing device 140 may further determine the blood flow information based on the medical images (e.g., the PET image frames) using another target model (i.e., the blood flow determination model) that has been trained using training samples, each of which including label blood flow information collected using the second tracer.

In this way, since the blood flow determination model and the cardiac function determination model may respectively learn the optimal mechanism for blood flow information determination and cardiac function information determination based on a large amount of data, the blood flow information and the cardiac function information determined using the blood flow determination model and the cardiac function determination model may be relatively more accurate.

As described elsewhere in the present disclosure, in some embodiments, the target model (e.g., the target model A, the target model B) may be used to determine both blood flow information and cardiac function information of the heart of the target subject, and such target model may be also referred to as the multitask model. Accordingly, in some embodiments, the blood flow information and cardiac function information of the heart may be determined via the same target model (e.g., the multitask model).

For example, FIG. 8 is a schematic diagram illustrating an exemplary process for determining blood flow information and cardiac function information via a multitask model according to some embodiments of the present disclosure. As shown in FIG. 8, the model input may be input into the multitask model. The multitask model may output both the blood flow information and the cardiac function information of the heart.

In some embodiments, if the target model obtained in operation 420 is the multitask model, the label information described in operation 420 may include label blood flow information and label cardiac function information, and both the blood flow information and the cardiac function information described in operation 430 may be determined based on the medical images and the multitask model.

In this way, the multitask model can simultaneously determine the blood flow information and the cardiac function information, thereby improving the efficiency of the determination of the blood flow information and the cardiac function information.

After the first tracer (e.g., the FDG) is injected into the target subject, it will pass through the blood pool and the myocardial successively. As described elsewhere in the present disclosure, since metabolites of the first tracer metabolized in the myocardium of the heart can not be absorbed by the myocardium totally (i.e., remain in the myocardium), when the first tracer concentrates in the myocardium, data collected by the first trace is less suitable for determining blood flow information and/or cardiac function information of the heart.

In some embodiments, the processing device 140 may divide the PET image frames into a first subset corresponding to a first stage of the medical scan and a second subset corresponding to a second stage of the medical scan. The first tracer may be more concentrated in the blood pool of the heart than the myocardium of the heart in the first stage, and the first tracer may be more concentrated in the myocardium than the blood pool in the second stage. In some embodiments, the first stage may be a period having a certain duration (e.g., 2 minutes, 3 minutes, etc.) after the first tracer is injected into the target subject, and the second stage may be a period in the medical scan other than the first stage. In some embodiments, the processing device 140 may divide the PET image frames into the first subset and the second subset based on the concentration of the first tracer in the blood pool and the myocardium. For example, a region in a PET image frame having a greater brightness corresponds to a position of the subject having a higher concentration of the first tracer. For each PET image frame, the processing device 140 may compare the brightness of a region corresponding to the blood pool (also referred to as a blood pool region) and the brightness of a region corresponding to the myocardial (also referred to as a myocardial region). If the blood pool region is brighter than the myocardial region in the PET image frame, the processing device 140 may determine that the PET image frame belongs to the first subset. If the myocardial region is brighter than the blood pool region in the PET image frame, the processing device 140 may determine that the PET image frame belongs to the second subset.

Further, the processing device 140 may determine a first portion of the blood flow information by processing the first subset using at least one pharmacokinetic model. For example, the processing device 140 may construct a first pharmacokinetic model for determining blood flow information using a quantitative analysis tool for molecular imaging (e.g., the PMOD software). Further, the processing device 140 may determine the first portion of the blood flow information by processing the first subset using the first pharmacokinetic model. Then, the processing device 140 may determine a second portion of the blood flow information by processing the second subset using the target model (e.g., the target model A or the target model B). The processing device 140 may combine the first portion and the second portion of the blood flow information to obtain the blood flow information of the target subject.

Since the first tracer is more concentrated in the blood pool of the heart than the myocardium of the heart in the first stage, the first portion of the blood flow information determined based on the first subset have desired accuracy even using at least one pharmacokinetic model. Since the first tracer is more concentrated in the myocardium of the heart than the blood pool of the heart in the first stage, the second subset of the PET image frames need to be processed by the target model (which is trained using data collected by the second tracer), so that the determined second portion of the blood flow information have desired accuracy. Thus, the obtained blood flow information may be accurate. Moreover, in this way, since the target model only needs to process a part of the medical images (i.e., the second subset), the data processing amount of the target model may be reduced, thereby improving the efficiency of the determination of blood flow information.

In some embodiments, the gated images may include a first group of gated images corresponding to the first stage of the medical scan and a second group of gated images corresponding to the second stage of the medical scan. For each of one or more candidate gated images of the first group of gated images, the processing device 140 may determine a left ventricular volume of the target subject corresponding to the candidate gated image. Specifically, the processing device 140 may determine one or more candidate gated images from the first group of gated images according to the heartbeat phases of the gated images. The one or more candidate gated images may include the gated image corresponding to an end-systole of the heart, the gated image corresponding to an end-diastole of the heart, etc. For each candidate gated image, the processing device 140 may segment the left ventricle of the heart from the candidate gated image, and determine the left ventricular volume of the target subject corresponding to the candidate gated image based on the segmentation result. Further, the processing device 140 may determine the first portion of the cardiac function information based on the one or more left ventricular volumes corresponding to one or more candidate gated images. For example, the processing device 140 may determine the left ventricular volume corresponding to the candidate gated image that corresponds to the end-systole of the heart as the ESV. As another example, the processing device 140 may determine the left ventricular volume corresponding to the gated image that corresponds to the end-diastole of the heart as the EDV. As still another example, the processing device 140 may determine the EF based on the EDV and ESV according to Equation (1) as below:

$$EF = \frac{( EDV - ESV )}{EDV} \times 100 \% , \qquad ( 1 )$$

Then, the processing device 140 may determine a second portion of the cardiac function information by processing the second group of gated images using the target model (e.g., the target model A or the target model B). The processing device 140 may combine the first portion and the second portion of the cardiac function information to obtain the cardiac function information of the target subject.

In this way, since the target model only needs to process a part of the gated images (i.e., the second group of gated images), the data processing amount of the target model may be reduced, thereby improving the efficiency of the determination of cardiac function information.

In 440, the processing device 140 (e.g., the determination module 320) may determine myocardial metabolic information of the heart by processing the medical images using a second pharmacokinetic model.

The myocardial metabolic information may indicate a state of the myocardial of the target subject. For example, the myocardial metabolic information may include a myocardial metabolic rate, etc. The second pharmacokinetic model may be a pharmacokinetic model for determining myocardial metabolic information.

As described in operation 410, the first tracer may be metabolized in the myocardium of the heart. Thus, the first tracer may be used to determine accurate myocardial metabolic information. In some embodiments, the processing device 140 may obtain the second pharmacokinetic model from a storage device (e.g., the storage device 150), and determine the myocardial metabolic information of the heart by processing the medical images (e.g., the PET image frames) using the second pharmacokinetic model.

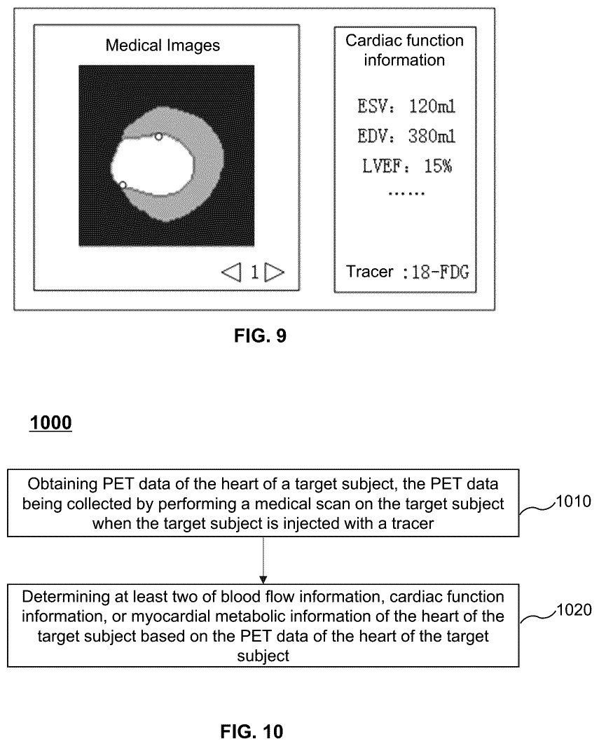

In some embodiments, the blood flow information, the cardiac function information, or the myocardial metabolic information of the heart may be sent to a terminal device (e.g., the terminal device 130) for display. For example, FIG. 9 is a schematic diagram illustrating exemplary cardiac function information of a subject according to some embodiments of the present disclosure. As shown in FIG. 9, medical images of the subject may be displayed on the left of the terminal device. The cardiac function information and tracer may be displayed on the right of the terminal device. The medical images include the left ventricular (LV) of the heart of the subject. The ESV, the EDV, and LVEF are 120 ml, 380 ml, and 15%, respectively. The tracer is 18F-FDG.

As described elsewhere in the present disclosure, conventionally, two PET scans with two different tracers need to be performed on a subject to obtain accurate and complete myocardial information, which is inefficient and unfriendly to the subject.

According to the systems and methods of the present disclosure, a target model is trained using label information collected by the second tracer, and used to determine blood flow information and cardiac function information based on medical images collected with the first tracer. The target model can learn the optimal mechanism for blood flow information determination and/or cardiac function information determination based on a large amount of data, thus, the blood flow information and/or the cardiac function information determined using the target model may be accurate. For example, by using the target model, medical images collected with FDG can be used to determine accurate blood flow information and cardiac function information. FDG is easy to obtain and has a low cost, and the medical images collected with FDG can be determine myocardial metabolic information. Compared with the conventional approach, the systems and methods of the present discloser may obtain accurate and comprehensive myocardial information by only performing one medical scan, which may improve efficiency of cardiac scanning and analysis.

FIG. 10 is a flowchart illustrating an exemplary process for myocardial information determination according to some embodiments of the present disclosure.

In 1010, the processing device 140 (e.g., the obtaining module 310) may obtain PET data of the heart of a target subject, the PET data being collected by performing a medical scan on the target subject when the target subject is injected with a tracer.

In some embodiments, the tracer includes the first tracer described in operation 410. In some embodiments, the first tracer may include FDG (e.g., 18F-FDG), acetic acid, or the like.

In some embodiments, the processing device 140 may obtain the PET data from one or more components (e.g., the PET scanner 110, the storage device 150) of the PET system 100 or an external source via a network (e.g., the network 120).

In 1020, the processing device 140 (e.g., the determine module 320) may determine at least two of blood flow information, cardiac function information, or myocardial metabolic information of the heart of the target subject based on the PET data of the heart of the target subject.

In some embodiments, the PET data may be collected by performing the medical scan on a region of interest (ROI) of the target subject via the PET scanner 110 over a scan time period. The target subject may include a patient, an animal, a phantom, or a portion thereof. The ROI may include the heart (or a portion thereof, e.g., the left ventricular) of the target subject.

In some embodiments, a first portion of the PET data for determining the blood flow information may be collected in a first time period of the scan time period from when the tracer is injected.

In some embodiments, the first time period or a second time period of the scan time period for collecting a second portion of the PET data are smaller than a third time period of the scan time period for collecting a third portion of the PET data. The second portion of the PET data may be used to determine the cardiac function information, the third portion of the PET data may be used to determine the myocardial metabolic information. In some embodiments, the second time period may be any one time period of the scan time period. In some embodiments, if the scan time period is smaller than a first time threshold (e.g., 15 minutes), the third time period may be a time period from when the tracer is injected. If the scan time period is greater than a second time threshold (e.g., 60 minutes), the third time period may be a time period including when the medical scan is finished.

In some embodiments, the processing device 140 may determine the myocardial metabolic information and at least one of the blood flow information and the cardiac function information based on the PET data of the heart of the target subject.

In some embodiments, the processing device 140 may generate medical images of the heart of the target subject based on the PET data of the heart of the target subject. Further, the processing device 140 may obtain a target model that has been trained using training samples, each of which includes label information collected using a second tracer. Further, the processing device 140 may determine the blood flow information and/or the cardiac function information of the heart of the target subject based on the medical images and the target model. More descriptions regarding the determination of the blood flow information and/or the cardiac function information of the heart of the target subject based on the medical images and the target model may be found elsewhere in the present disclosure (e.g., FIGS. 4-8 and the descriptions thereof).

In some embodiments, as described in operation 410, the medical images reconstructed based on the sets of PET data may include gated images corresponding to the multiple heartbeat phases. The processing device 140 may determine the cardiac function information of the heart of the target subject based on the gated images. Specifically, the processing device 140 may determine one or more candidate gated images from the gated images according to the heartbeat phases of the gated images. The one or more candidate gated images may include the gated image corresponding to an end-systole of the heart, the gated image corresponding to an end-diastole of the heart, etc. For each candidate gated image, the processing device 140 may segment the left ventricle of the heart from the candidate gated image, and determine the left ventricular volume of the target subject corresponding to the candidate gated image based on the segmentation result. Further, the processing device 140 may determine the cardiac function information based on the one or more left ventricular volumes corresponding to one or more candidate gated images. For example, the processing device 140 may determine the left ventricular volume corresponding to the candidate gated image that corresponds to the end-systole of the heart as the ESV. As another example, the processing device 140 may determine the left ventricular volume corresponding to the gated image that corresponds to the end-diastole of the heart as the EDV. As still another example, the processing device 140 may determine the EF based on the EDV and ESV according to Equation (2) as below:

$$EF = \frac{( EDV - ESV )}{EDV} \times 100 \% , \qquad ( 2 )$$

In this way, when the myocardial blood supply is insufficient, accurate left ventricle segmentation can still be obtained, thereby the left ventricular volumes determined based on the gated images having greatly accuracy. Thus, the obtained cardiac function information may be accurate.

It should be noted that the above descriptions about the processes 400 and 1000 are merely provided for illustration purposes, and not intended to be limiting. For persons having ordinary skills in the art, multiple variations and modifications may be made under the teachings of the present disclosure. However, those variations and modifications do not depart from the scope of the present disclosure. For example, the operations of the illustrated processes 400 and 1000 are intended to be illustrative. In some embodiments, the processes 400 and 1000 may be accomplished with one or more additional operations not described, and/or without one or more of the operations discussed. Additionally, the order in which the operations of the processes 400 and 1000 and regarding descriptions are not intended to be limiting. As another example, operation 440 may be omitted. As still another example, referring to FIG. 6, the processing device 140 may determine the blood flow information and/or the cardiac function information based on the pseudo images using the first pharmacokinetic model and/or the second pharmacokinetic model instead of the target model B.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A system, comprising:
at least one storage device including a set of instructions; and
at least one processor in communication with the at least one storage device, wherein when executing the set of instructions, the at least one processor is configured to direct the system to perform operations including:
obtaining medical images of the heart of a target subject, the medical images being collected by performing a medical scan on the target subject when the target subject is injected with a first tracer;
obtaining a target model that has been trained using training samples, each of which includes label information collected using a second tracer; and
determining blood flow information and/or cardiac function information of the heart of the target subject based on the medical images and the target model.

2. The system of claim 1, wherein
the first tracer includes fludeoxyglucose (FDG) or acetic acid; or
the second tracer includes 13N-ammonia (NH3) or Rubidium 82 (Rb82).

3. The system of claim 1, wherein when the target model is used to determine the blood flow information, the medical images include PET image frames that are consecutively acquired in the medical scan.

4. The system of claim 3, wherein a model input of the target model includes:
segmentation images of the heart by segmenting the heart from the PET image frames; or
time-activity curves of physical points at the heart.

5. The system of claim 1, wherein when the target model is used to determine the cardiac function information, the medical images include gated images generated based on a heartbeat signal of the heart collected in the medical scan.

6. The system of claim 5, wherein a model input of the target model includes segmentation images of the heart by segmenting the heart from the gated images.

7. The system of claim 1, wherein each training sample of the target model further includes a training input and is determined by:
obtaining first sample medical images and second sample medical images of a sample subject, the first sample medical images being collected by performing a first sample medical scan on the sample subject when the sample subject is injected with the first tracer, and the second sample medical images being collected by performing a second sample medical scan on the sample subject when the sample subject is injected with the second tracer;
determining the label information based on the second sample medical images; and
determining the training input based on the first sample medical images.

8. The system of claim 1, wherein the determining blood flow information and/or cardiac function information of the heart of the target subject based on the medical images and the target model comprises:
for each of the medical images, generating a pseudo image corresponding to the second tracer by processing the medical image using an image transformation model, the image transformation model being a trained machine learning model;
determining a model input based on the pseudo image of each medical image;
determining the blood flow information and/or the cardiac function information by inputting the model input into the target model.

9. The system of claim 8, wherein each training sample of the target model further includes a training input and is determined by:
obtaining second sample medical images of a sample subject, the second sample medical images being collected by performing a second sample medical scan on the sample subject when the sample subject is injected with the second tracer; and
determining the training input and the label information based on the second sample medical images.

10. The system of claim 3, wherein the determining blood flow information and/or cardiac function information of the heart of the target subject based on the medical images and the target model comprises:
dividing the PET image frames into a first subset corresponding to a first stage of the medical scan and a second subset corresponding to a second stage of the medical scan, the first tracer being more concentrated in the blood pool of the heart than the myocardium of the heart in the first stage, the first tracer being more concentrated in the myocardium than the blood pool in the second stage;
determining a first portion of the blood flow information by processing the first subset using at least one pharmacokinetic model; and
determining a second portion of the blood flow information by processing the second subset using the target model.

11. The system of claim 5, wherein
the gated images include a first group of gated images corresponding to a first stage of the medical scan and a second group of gated images corresponding to a second stage of the medical scan, the first tracer being more concentrated in the blood pool of the heart than the myocardium of the heart in the first stage, the first tracer being more concentrated in the myocardium than the blood pool in the second stage;
the determining blood flow information and/or cardiac function information of the heart of the target subject based on the medical images and the target model comprises:
for each of one or more candidate gated images of the first group of gated images, determining a left ventricular volume of the target subject corresponding to the candidate gated image;
determining a first portion of the cardiac function information based on the one or more left ventricular volumes corresponding to one or more candidate gated images; and
determining a second portion of the cardiac function information by processing the second group of gated images using the target model.

12. The system of claim 1, wherein
the label information includes label blood flow information,
the blood flow information is determined based on the medical images and the target model, and
the cardiac function information is determined based on the medical images using another target model that has been trained using training samples, each of which including label cardiac function information collected using the second tracer.

13. The system of claim 1, wherein
the label information includes label cardiac function information,
the cardiac function information is determined based on the medical images and the target model, and
the blood flow information is determined based on the medical images using another target model that has been trained using training samples, each of which includes label blood flow information collected using the second tracer.

14. The system of claim 1, wherein
the label information includes label blood flow information and label cardiac function information,
the target model is a multitask model, and
both the blood flow information and the cardiac function information are determined based on the medical images and the multitask model.

15. The system of claim 1, wherein the operations further comprise:
determining myocardial metabolic information of the heart by processing the medical images using a second pharmacokinetic model.

16. A system, comprising:
at least one storage device including a set of instructions; and
at least one processor in communication with the at least one storage device, wherein when executing the set of instructions, the at least one processor is configured to direct the system to perform operations including:
obtaining PET data of the heart of a target subject, the PET data being collected by performing a medical scan on the target subject when the target subject is injected with a tracer; and
determining at least two of blood flow information, cardiac function information, or myocardial metabolic information of the heart of the target subject based on the PET data of the heart of the target subject.

17. The system of claim 16, wherein a first portion of the PET data for determining the blood flow information is collected in a first time period from when the tracer is injected.

18. The system of claim 16, wherein a first time period for collecting a first portion of the PET data or a second time period for collecting a second portion of the PET data are smaller than a third time period for collecting a third portion of the PET data, the first portion of the PET data being used to determine the blood flow information, the second portion of the PET data being used to determine the cardiac function information, the third portion of the PET data being used to determine the myocardial metabolic information.

19. The system of claim 16, wherein the determining at least two of blood flow information, cardiac function information, or myocardial metabolic information of the heart of the target subject based on the PET data of the heart of the target subject includes:
generating medical images of the heart of the target subject based on the PET data of the heart of the target subject;
obtaining a target model that has been trained using training samples, each of which includes label information collected using a second tracer; and
determining the blood flow information and/or the cardiac function information of the heart of the target subject based on the medical images and the target model.

20-34. (canceled)

35. A method, the method being implemented on a computing device having at least one storage device and at least one processor, the method comprising:
obtaining medical images of the heart of a target subject, the medical images being collected by performing a medical scan on the target subject when the target subject is injected with a first tracer;
obtaining a target model that has been trained using training samples, each of which includes label information collected using a second tracer; and
determining blood flow information and/or cardiac function information of the heart of the target subject based on the medical images and the target model.

36-42. (canceled)
